(19) United States
(12) Patent Application Publication
(10) Pub. No.: US 2007/0259026 A1
(43) Pub. Date: Nov. 8, 2007

(54) Vasodialating dressing for use with intravenous catheters

(76) Inventors: Robert Ang, New York, NY (US); David Geliebter, Franklin Lakes, NJ (US)

Correspondence Address: THORPE NORTH & WESTERN, LLP., 8180 SOUTH 700 EAST, SUITE 350, SANDY, UT, 84070, US

(21) Appl. No.: 11/788,146

(22) Filed: Apr. 18, 2007

Related U.S. Application Data
(60) Provisional application No. 60/794,218, filed on Apr. 20, 2006.

(51) Int. Cl. A61L 15/00; A61K 31/21

(52) U.S. Cl. 424/445; 514/509

(57) ABSTRACT

The present invention is drawn to adhesive dressings and associated systems and methods for use in reducing the occurrence of phlebitis and extending the lifespan of intravenous catheters. The adhesive dressings can include a backing layer, and a nitroglycerin-containing composition supported at least in part by the backing layer. The adhesive dressing can have a drug delivery zone defined by the area where the nitroglycerin-containing composition contacts an intact human skin site. The drug delivery zone can have an area of at least 2.5 cm² and can be configured such that when the adhesive dressing is applied to secure an intravenous catheter the drug delivery zone substantially overlays a vessel in which the intravenous catheter is inserted. The nitroglycerin-containing composition can be formulated to deliver nitroglycerin at from about 5 μg/hour to about 80 μg/hour.

This application claims the benefit of U.S. Provisional Patent Application No. 60,794,218, filed Apr. 20, 2006, which is incorporated herein by reference in its entirety.

### FIELD OF THE INVENTION

The present invention is drawn to dressings. More particularly, the present invention is drawn to dressings for administering active agents that reduce incidences of phlebitis associated with insertion and use of intravenous catheters.

### BACKGROUND OF THE INVENTION

Intravenous therapy is a common method of treatment for hospitalized patients. It is estimated that approximately 80% of hospitalized patients receive either continuous or intermittent intravenous therapy on a daily basis. A common cause of failure for intravenous therapy is phlebitis, or inflammation of a vessel. Phlebitis is believed to be caused, in part, by venoconstriction at the infusion site brought about by irritation of the endothelium. It is reported that phlebitis occurs in between 30% to 70% of all infusions. The prevention or reduction of the occurrence of phlebitis would be extremely valuable, saving patients from pain and discomfort and reducing the number of catheters which are replaced.

### SUMMARY OF THE INVENTION

The present invention is drawn to adhesive dressings and associated systems designed to reduce the occurrence of phlebitis and increase the available lifetime of intravenous catheters. In one embodiment, an adhesive dressing for securing intravenous catheters is provided. The adhesive dressing comprises a backing layer and a nitroglycerin-containing composition supported at least in part by the layer. The adhesive dressing has a drug delivery zone defined by the area where the nitroglycerin-containing composition contacts an intact human skin site. In certain specific embodiments the drug delivery zone can have an area of at least 2.5 cm² and/or can be configured such that when the adhesive dressing is applied to secure an intravenous catheter, the drug delivery zone substantially overlays a vessel in which the intravenous catheter is inserted. The nitroglycerin-containing composition can be formulated to deliver nitroglycerin at from about 5 μg/hour to about 80 μg/hour.

In another embodiment, an adhesive dressing for use in securing intravenous catheters includes a backing layer and a nitroglycerin-containing composition supported at least in part by the backing layer. The adhesive dressing can have a drug delivery strip defined by the area where the nitroglycerin-containing composition contacts an intact human skin site. The drug delivery strip can have an area of between 5 to 25 cm² and can be laterally flanked by the portions of the backing layer coated with a nitroglycerin-free adhesive. The drug delivery strip can be configured such that when the adhesive dressing is applied to secure an intravenous catheter, the drug delivery strip substantially overlays a vessel in which the intravenous catheter is inserted and the adhesive coated portions of the backing layer run substantially parallel to, i.e. parallel or approximately parallel, and over the vessel in which the catheter is inserted. The nitroglycerin-containing composition can be formulated to deliver nitroglycerin at from about 5 μg/hour to about 80 μg/hour.

In yet another embodiment, a system for preventing or reducing the occurrence of phlebitis in a patient is provided. The system includes a catheter configured to be intravenously located in a vessel of the patient and an adhesive dressing capable of securing an intravenously located catheter. The adhesive dressing comprises a backing layer, and a nitroglycerin-containing composition supported at least in part by the layer. The adhesive dressing has a drug delivery zone defined by the area where the nitroglycerin-containing composition contacts an intact human skin site. The drug delivery zone can have an area of at least 2.5 cm² and can be configured such that when the adhesive dressing is applied to secure an intravenous catheter the drug delivery zone substantially overlays a vessel in which the intravenous catheter is inserted. The nitroglycerin-containing composition can be formulated to deliver nitroglycerin at from about 5 μg/hour to about 80 μg/hour.

### BRIEF DESCRIPTION OF THE DRAWINGS

In each of the FIGS., crosshatching is used to depict the location of the active portion of the dressing, i.e. the location where nitroglycerin is present as part of the adhesive matrix.

FIG. 1 depicts an exemplary embodiment of a nitroglycerin dressing in which the active portion of the dressing is the entire surface area.

FIG. 2 depicts another exemplary embodiment of a nitroglycerin dressing in which the active portion of the dressing is less than the entire surface area.

FIG. 3 depicts another exemplary embodiment of a nitroglycerin dressing in which the active portion of the dressing is a strip that is alignable with a vessel.

FIG. 4 depicts another exemplary embodiment of a nitroglycerin dressing in which the active portion of the dressing is a triangular shape.

FIG. 5 depicts an exemplary embodiment of a nitroglycerin dressing having a notched shape to accommodate an IV catheter.

### DETAILED DESCRIPTION OF THE PREFERRED EMBODIMENT(S)

Before particular embodiments of the present invention are disclosed and described, it is to be understood that this invention is not limited to the particular process and materials disclosed herein and as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and, “the” include plural referents unless the context clearly dictates otherwise. Thus, for example, reference to a drug-containing composition including “an adhesive dressing” includes one or more adhesive dressing(s) and reference to “the drug delivery zone” includes reference to one or more drug delivery zone(s).

As used herein, “subject” refers to a mammal that may benefit from the administration of the dressings, systems, or methods of this invention. Examples of subjects include humans, and may also include other animals such as horses, pigs, cattle, other farm animals, dogs, cats, rabbits, aquatic mammals, etc.

As used herein, the terms “formulation” and “composition” are used interchangeably and refer to situations where there are two or more compounds, elements, or molecules in a common system.

As used herein, the term “vessel” refers to blood transporting components of a circulatory system, including arteries and veins.

As used herein, the terms “drug delivery zone” refers to the area of skin which comes into direct contact with the portion of the adhesive dressing which delivers the nitroglycerin. Drug delivery zones can cover an entire adhesive patch surface area, or can be less than the entire adhesive area of the patch. In one embodiment, the drug delivery zone can be a drug “delivery strip” that is flanked laterally by adhesive, non-drug delivery zones.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

Nitroglycerin is a vasodilator which acts by relaxing vascular smooth muscle, which results in arteriolar, arterial, and venous dilation. Nitroglycerin has been formulated into transdermal patches for use in treating systemic cardiovascular ailments such as angina, congestive heart failure, and hypertension. The transdermal patches for use in treating these systemic cardiovascular ailments typically deliver nitroglycerin to a subject at from about 5 mg/day to about 20 mg/day. The present invention provides for a nitroglycerin-containing adhesive dressing for securing intravenous catheters (IV) and preventing or reducing the incidence of phlebitis thereby extending the lifespan of the IV. In addition to peripheral IV catheters, the nitroglycerin delivering adhesive dressing can be used for other types of access: venous or arterial, peripheral or central (or peripherally inserted central), and dialysis ports or fistulas. As discussed above, nitroglycerin is believed to promote venodilation, thereby countering the venoconstriction that is thought to be the primary cause of phlebitis at IV puncture sites. The adhesive dressings of the present invention may be applied before placement of the IV in order to dilate the target vessel to simplify placement, simultaneously with the insertion of the IV, or after the IV is placed.

The present invention provides for an adhesive dressing capable of securing an intravenous catheter and delivering a low dosage amount of nitroglycerin to the patient from the adhesive dressing. The adhesive dressings generally include a backing layer and a nitroglycerin-containing composition which is supported at least in part by the backing layer. The adhesive dressing has a drug delivery zone or strip which is defined by the areas where the nitroglycerin-containing composition contacts an intact human skin site when the adhesive dressing is applied. The drug delivery zone has a minimum area of 2.5 cm² and can be configured such that when the adhesive dressing is applied a skin site to secure an intravenous catheter, at least a portion of the drug delivery zone overlays the vessel in which the intravenous catheter is inserted. Depending on a number of variables, including the size of the drug delivery zone, the concentration of nitroglycerin, etc., the nitroglycerin-containing composition can be formulated to deliver nitroglycerin from the drug delivery zone of the adhesive dressing at a rate of from about 5 μg/hr to about 80 μg/hr.

Another embodiment of the present invention provides a system for preventing or reducing the occurrence of phlebitis in a patient. The system includes a catheter configured to be intravenously located in a vessel of the patient and an adhesive dressing capable of securing an intravenously located catheter. The adhesive dressing can generally include a backing layer and a nitroglycerin-containing composition which is supported at least in part by the backing layer. The adhesive dressing has a drug delivery zone or strip which is defined by the areas where the nitroglycerin-containing composition contacts an intact human skin site when the adhesive dressing is applied. The drug delivery zone has a minimum area of 2.5 cm² and can be configured such that when the adhesive dressing is applied to a skin site to secure an intravenous catheter, at least a portion of the drug delivery zone overlays the vessel in which the intravenous catheter is inserted.

In another embodiment a, a system for preventing or reducing the occurrence of phlebitis in a patient is provided which includes an catheter and an adhesive dressing which are integrated together into a single device. The adhesive dressing comprises a backing layer, and a nitroglycerin-containing composition supported at least in part by the layer. The adhesive dressing has a drug delivery zone defined by the area where the nitroglycerin-containing composition contacts an intact human skin site. The drug delivery zone can have an area of at least 2.5 cm² and can be configured such that when the adhesive dressing is applied to secure an intravenous catheter the drug delivery zone substantially overlays a vessel in which the intravenous catheter is inserted. The nitroglycerin-containing composition can be formulated to deliver nitroglycerin at from about 5 μg/hour to about 80 μg/hour.

In each of the above described system embodiments, the catheter and the adhesive dressing can be co-packaged into a kit for ease of distribution and use.

The adhesive dressings of the present invention can come in a variety of sizes and shapes depending on a several factors such as the desired location of the intravenous catheter to be secured. In one embodiment, the adhesive dressing can have an area of from about 50 cm² to about 100 cm². The shape of the dressing can also be varied. The adhesive dressings can be rectangular, square, triangular, circular, elliptical, oval, butterfly shaped, or any other shape which is capable of securing an intravenous catheter and delivering a dose of nitroglycerin to the skin area proximate, preferably above, the catheterized vessel. It may be shaped so as to substantially avoid covering hair when placed in typical IV access sites (for example, long and thin to fit over less hairy portions of the forearm where arterial (such as radial) or venous access is obtained. The adhesive dressing can also have slits or notches so as to better accommodate catheters.

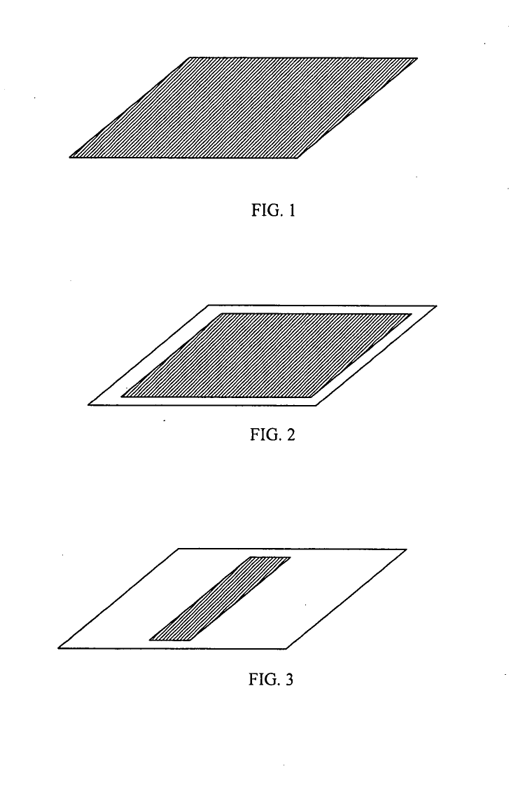

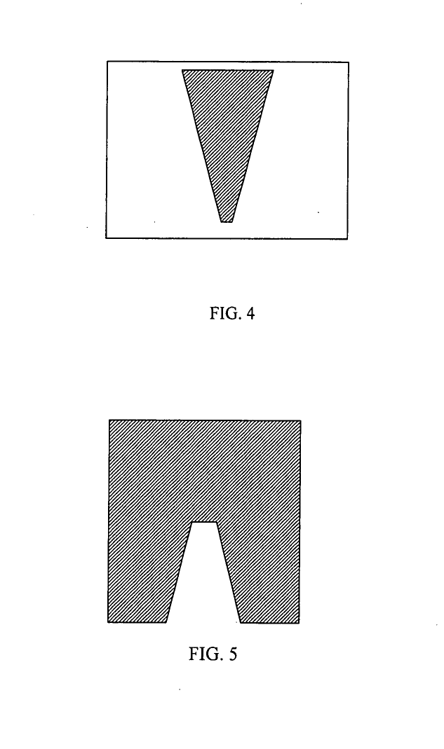

More specifically, the size (area), and shape of the drug delivery zones of the adhesive dressings can be varied depending on the shape of the adhesive dressing as well as the desired delivery rates of nitroglycerin. FIGS. 1 and 2 show embodiments in which the drug delivery zone has an area which is substantially equivalent to the area of the adhesive dressing contacting the skin, or just slightly smaller. In these and other embodiments, the drug delivery zone can have an area of from about 2.5 cm² to about 100 cm². In yet another embodiment, the drug delivery zone can have an area of from about 3 cm² to about 50 cm². In still another embodiment, the drug delivery zone can have an area of from about 5 cm² to about 25 cm². In addition to the structures shown in FIGS. 1 and 2, the drug delivery zones or strips can take on various shapes and orientations with respect to the backing layer of the adhesive dressing. The drug delivery zones can be generally rectangular, triangular, or circular in shape. For example, FIG. 3 shows an adhesive dressing of the present invention in which the drug delivery zone is a drug delivery strip. The drug delivery strip is laterally flanked on both sides by regions of backing layer coated non-nitroglycerin-containing adhesive. The adhesive dressing is configured such that the drug delivery strip substantially overlays the catheterized vessel while the nitroglycerin free adhesive coated regions of the backing layer run substantially parallel to the vessel in which the catheter is inserted. FIG. 4 shows an embodiment of the present invention similar to the embodiment shown in FIG. 3. The drug delivery strip in the FIG. 4 embodiment is triangular in shape but the drug delivery strip is similarly designed with laterally flanking nitroglycerin free adhesive regions. When the drug delivery zone is triangular in shape, the dressing can be positioned so that the apex of the triangle overlies or is near the insertion site. The triangular shape fans out from the insertion site to cover the vessel should the vessel be tortuous or if the dressing is not precisely located. The triangular shape may be truncated (i.e., the triangle has one or more blunt tips instead of sharp tips). Advantages of using a nitroglycerin-containing adhesive strip over the catheterized vessel while other areas of the adhesive that laterally flank the nitroglycerin-containing adhesive strip are free of the nitroglycerin can be related to dosage delivery, more localized drug delivery, providing enhanced adhesion, increasing the surface area of attachment without requiring drug over the entire delivery zone, etc. In another embodiment, FIG. 5 shows a notched intravenous dressing. The dressing shown in FIG. 5 has a drug delivery zone substantially equivalent to the total area of the adhesive dressing.

The adhesive dressing of the present invention can be used to secure the intravenous catheter and deliver steady amounts of nitroglycerin for periods of time from 4 hours to 7 days. In one embodiment, the nitroglycerin-containing composition of the adhesive dressing is formulated to deliver nitroglycerin for a period of from 1 day to 3 days. In another embodiment, the nitroglycerin-containing composition of the adhesive dressing is formulated to deliver nitroglycerin for a period of from 12 hours to 24 hours.

The backing layer used in the adhesive dressings can be made of a variety of medically acceptable materials. The backing layer can be generally flexible enough to be applied to the contours of a skin site yet strong enough to adequately secure a catheter. In some embodiments, the backing layer can be translucent. In other embodiments, the backing layer can be transparent. Transparent and translucent backing layers can be advantageous because of the ability to still see the site at which the catheter is inserted into the vessel.

When the nitroglycerin-containing composition does not coat substantially all of the backing layer, the uncoated regions of the backing layer can be coated with medically acceptable adhesives. Medically acceptable adhesives are well known in the art. Nitroglycerin is known to have some adhesive properties and can act as the loan adhesive in some embodiments where the backing layer is substantially coated with the nitroglycerin-containing composition. The concentration of nitroglycerin in a nitroglycerin-containing composition can dramatically affect the adhesive properties of the composition. The nitroglycerin-containing composition can also include a second or supplementary adhesive. This is particularly advantageous when the nitroglycerin-containing composition coats substantially all of the backing layer of the adhesive dressing.

The nitroglycerin-containing compositions of the present invention can be formulated to deliver nitroglycerin from the drug delivery zone at rates of from about 5 μg/hr to about 80 μg/hr. In one embodiment the nitroglycerin-containing composition can be formulated to deliver from about 10 μg/hr to about 60 μg/hr. The nitroglycerin-containing compositions can also be formulated so that they provide delivery rates at the drug delivery zone of from about 1 μg/cm²/day to 600 μg/cm²/day. In another embodiment the nitroglycerin-containing composition on the adhesive dressing can be formulated to provide delivery rates of from about 10 μg /cm²/day to about 400 μg/cm²/day. In yet another embodiment, the nitroglycerin-containing composition is formulated to provide delivery rates of from 50 μg/cm²/day to about 300 μg/cm²/day.

In addition to nitroglycerin, the nitroglycerin-containing composition of the present invention can also include various binders and excipients as are well known in the art of transdermal drug delivery. Examples include, but are not limited to solvents, permeation enhancers, crosslinkers, and other active compounds, such as non-steroidal anti-inflammatory compounds (NSAIDS). Examples of permeation enhancers include but are not limited to polyethylene glycols, surfactants, and combinations thereof.

### EXAMPLES

The following examples illustrate exemplary embodiments of the invention. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what is presently deemed to be practical embodiments of the invention.

### Examples 1-3

Prototype nitroglycerin-containing composition formulations for use in the adhesive dressings of the present invention are as follows. Several nitroglycerin-containing compositions are prepared in accordance with embodiments of the present invention with the starting components listed in Table 1.

TABLE 1

| | | | | DuroTak |
|---|---|---|---|---|
| | | | Ethyl | 87-2194 |
| | Dose | Nitroglycerin | Acetate | (liquid) |
| Example | (μg/hr) | (Wt. %) | (Wt. %) | (Wt. %) |
| 1 | 17.1 | 2.6% | 7.7% | 89.8% |
| 2 | 23.3 | 3.4% | 10.1% | 86.6% |
| 3 | 42.9 | 4.9% | 14.7% | 80.4% |

The adhesive dressings of Examples 1-3 are prepared in the following manner:

1. Nitroglycerin is diluted in the DuroTak 87-2194 adhesive and ethyl acetate solvent forming the drug solution.

2. The drug solution of the adhesive blend is formed onto a release liner using a mechanical coater.

3. The coated release liner is then passed through an oven which causes the solvent to evaporate completely, forming a solid, tacky layer of adhesive matrix that contains nitroglycerin dispersed in a dry matrix.

4. The nitroglycerin-containing matrix is attached to a backing layer (polyethylene in some embodiments). The nitroglycerin-containing matrix can cover the substantially all of the backing layer, or only portions of the backing layer, such as in FIGS. 3 or 4. If the nitroglycerin-containing matrix covers only a portion of the backing layer, an adhesive, e.g., DuroTak or other suitable adhesive(s), can be coated on areas where the nitroglycerin-containing matrix is present.

The resulting nitroglycerin-containing compositions from Examples 1-3 have finished product content (Wt%) as shown in Table 2.

TABLE 2

| | | Total | Durotack 87-2194 |
|---|---|---|---|
| | Nitroglycerin | Nitroglycerin | (remaining solids) |
| Example | (Wt %) | (mg/cm²) | (Wt %) |
| 1 | 6% | 0.493 | 94% |
| 2 | 8% | 0.657 | 92% |
| 3 | 12% | 0.985 | 88% |

### Example 4

An adhesive dressing with a drug delivery zone of 25 cm² which delivers nitroglycerin at a rate of 17.1 μg/hr is applied to a forearm to secure an intravenous catheter. The drug delivery zone delivers nitroglycerin for a period of 24 hours reducing vein inflammation and inhibiting phlebitis. The adhesive dressing is removed after 24 hours and replaced with a new nitroglycerin-containing adhesive dressing.

### Example 5

Same as Example 4, except that adhesive dressing is capable of delivering nitroglycerin to the vein infusion site for a period of up to 7 days before being replaced.

### Example 6

A system for preventing or reducing the occurrence of phlebitis in a patient with having a catheter is employed. An intravenous catheter is placed in the arm of the patient and secured with an adhesive dressing having a size of 100 cm² and having a drug delivery zone of 50 cm². Nitroglycerin is delivered from the drug delivery zone at a rate of about 42.9 μg/hr for a period of up to 4 days. The drug delivery zone is triangular in shape (similar to the drug delivery zone shown in FIG. 4) and the adhesive dressing is placed on the arm so as to secure the catheter and substantially overlay the catheterized vein.

### Example 7

Same as Example 6, except that the adhesive dressing is a notched rectangle so as to allow easy access to the catheter.

### Example 8

A system for use in preventing or reducing the occurrence of phlebitis in a patient having a catheter is employed. The system includes a peripheral IV catheter and an adhesive dressing co-packaged together. The adhesive dressing is configured to be compatible with the system's peripheral IV catheter and has a size of 100 cm² and a drug delivery zone of 50 cm². The peripheral IV catheter is placed in the arm of the patient and secured using the adhesive dressing. The drug delivery zone of the adhesive dressing is formulated to deliver nitroglycerin at a rate of about 17.1 μg/hr for a period of up to 4 days. The drug delivery zone is triangular in shape (similar to the drug delivery zone shown in FIG. 4) and the adhesive dressing is placed on the arm so as to secure the catheter and substantially overlay the catheterized vein.

### Example 9

A system for use in preventing or reducing the occurrence of phlebitis in a patient having a catheter is employed. The system includes a peripherally inserted central catheter (PIC catheter) and an adhesive dressing which are integrated together in a single device. The PIC catheter is placed in the arm of the patient and secured using the integrated adhesive dressing. The drug delivery zone of the adhesive dressing is formulated to deliver nitroglycerin at a rate of about 23.3 μg/hr for a period of up to 7 days. The drug delivery zone is a rectangular in shaped strip (similar to the drug delivery zone shown in FIG. 3) and substantially overlays the catheterized vein.

### Example 10

A system for use in preventing or reducing the occurrence of phlebitis in a patient with an intravenous catheter is employed. The system includes a central venous catheter (CVC) and an adhesive dressing. The CVC is placed in the neck of the patient and secured using the integrated adhesive dressing. The drug delivery zone of the adhesive dressing is formulated to deliver nitroglycerin at a rate of about 30 μg/hr for a period of up to 7 days.

### Example 11

A system for use in preventing or reducing the occurrence of phlebitis in a patient having catheter is employed. The system includes an radial arterial catheter and an adhesive dressing. The arterial catheter is placed at the base of the thumb of a patient and secured using an adhesive dressing which was co-packaged with the arterial catheter. The drug delivery zone of the adhesive dressing is formulated to deliver nitroglycerin at a rate of about 28 μg/hr for a period of up to 3 days. The adhesive dressing is configured similarly to the dressing shown in FIG. 5.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. An adhesive dressing for use in securing intravenous catheters, comprising:
a backing layer, and
a nitroglycerin-containing composition supported at least in part by the backing layer, said adhesive dressing having a drug delivery zone defined by the area where the nitroglycerin-containing composition contacts an intact human skin site, said drug delivery zone having an area of at least 2.5 cm² and being configured such that when the adhesive dressing is applied to secure an intravenous catheter, the drug delivery zone overlays a vessel in which the intravenous catheter is inserted, said nitroglycerin-containing composition being formulated to deliver nitroglycerin at from about 5 μg/hour to about 80 μg/hour.

2. The adhesive dressing of claim 1, wherein the drug delivery zone has an area from about 2.5 cm² to 100 cm².

3. The adhesive dressing of claim 1, wherein the adhesive dressing has an area where the adhesive dressing contacts the skin surface, said area being from 50 cm² to 100 cm².

4. The adhesive dressing of claim 3, wherein the drug delivery zone has an area substantially equivalent to the area of the adhesive dressing.

5. The adhesive dressing of claim 1, wherein the nitroglycerin is present in the nitroglycerin-containing composition in an amount of from 6 wt % to 20 wt %.

6. The adhesive dressing of claim 1, wherein the nitroglycerin-containing composition is formulated to deliver the nitroglycerin for a period of from 4 hours to 7 days.

7. The adhesive dressing of claim 1, wherein the nitroglycerin-containing composition further includes a permeation enhancer selected from the group consisting of polyethylene glycols, surfactants, and combinations thereof.

8. The adhesive dressing of claim 1, wherein the nitroglycerin-containing composition provides a delivery rate of nitroglycerin at the drug delivery zone at from about 1 μg/cm²/day to about 600 μg/cm²/day.

9. The adhesive dressing of claim 1, wherein the drug delivery zone has a shape selected from the group consisting of: a rectangle, a triangle, a circle, and an oval.

10. The adhesive dressing of claim 1, wherein when the dressing is applied to a skin surface to secure a catheter in a vessel, the drug delivery zone overlays the vessel upstream from catheter.

11. An adhesive dressing for use in securing intravenous catheters, comprising:
a backing layer, and
a nitroglycerin-containing composition supported at least in part by the backing layer, said adhesive dressing having a drug delivery strip which is defined by the area where the nitroglycerin-containing composition contacts an intact human skin site, said drug delivery strip having an area between 5 cm² to 25 cm² and being laterally flanked by regions of the backing layer coated with a nitroglycerin-free adhesive, said drug delivery strip being configured such that when the adhesive dressing is applied to secure an intravenous catheter, the drug delivery strip substantially overlays a vessel in which the intravenous catheter is inserted while the nitroglycerin-free adhesive present on the backing layer laterally flanks the vessel in which the catheter is inserted, said nitroglycerin-containing composition being formulated to deliver nitroglycerin at from about 5 μg/hour to about 80 μg/hour.

12. The adhesive dressing of claim 11, wherein the adhesive dressing has an area where the adhesive dressing contacts the skin surface, said area being from 50 cm² to 100 cm².

13. The adhesive dressing of claim 11, wherein the nitroglycerin is present in the nitroglycerin-containing composition in an amount of from 6 wt % to 20 wt %.

14. The adhesive dressing of claim 11, wherein the nitroglycerin-containing composition is formulated to deliver the nitroglycerin for a period of from 4 hours to 7 days.

15. The adhesive dressing of claim 11, wherein the nitroglycerin-containing composition further includes a permeation enhancer selected from the group consisting of polyethylene glycols, surfactants, and combinations thereof.

16. The adhesive dressing of claim 11, wherein the nitroglycerin-containing composition provides a delivery rate of nitroglycerin at the drug delivery zone at from about 1 μg/cm²/day to about 600 μg/cm²/day.

17. The adhesive dressing of claim 11, wherein the drug delivery zone has a shape selected from the group consisting of: a rectangle, a triangle, a circle, and an oval.

18. The adhesive dressing of claim 11, wherein when the dressing is applied to a skin surface to secure a catheter in a vessel the drug delivery zone overlays the vessel upstream from catheter.

19. A system for use in preventing or reducing the occurrence of phlebitis in a catherterized subject, comprising:
a) a catheter configured to be intravenously located in a vessel of the subject; and
b) an adhesive dressing capable of securing the catheter when it is intravenously placed in the vessel of the subject, said adhesive dressing comprising:
a backing layer, and
a nitroglycerin-containing composition supported at least in part by the backing layer, said adhesive dressing having a drug delivery zone defined by the area where the nitroglycerin-containing composition contacts an intact human skin site, said drug delivery zone having an area of at least 2.5 cm² and being configured such that when the adhesive dressing is applied to secure an intravenous catheter the drug delivery zone overlays a vessel in which the intravenous catheter is inserted, said nitroglycerin-containing composition being formulated to deliver nitroglycerin at from about 5 μg/hour to about 80 μg/hour.

20. The system of claim 19, wherein the drug delivery zone has an area from about 2.5 cm² to 100 cm².

21. The system of claim 19, wherein the adhesive dressing has an area where the adhesive dressing contacts the skin surface, said area being from 50 cm² to 100 cm².

22. The system dressing of claim 21, wherein the drug delivery zone has an area substantially equivalent to the area of the adhesive dressing.

23. The system of claim 19, wherein the nitroglycerin is present in the nitroglycerin-containing composition in an amount of from 6 wt % to 20 wt %.

24. The system of claim 19, wherein the nitroglycerin-containing composition is formulated to deliver the nitroglycerin for a period of from 4 hours to 7 days.

25. The system of claim 19, wherein the nitroglycerin-containing composition further includes a permeation enhancer selected from the group consisting of polyethylene glycols, surfactants, and combinations thereof.

26. The system of claim 19, wherein the nitroglycerin-containing composition is formulated to deliver nitroglycerin at from 10 μg/hour to 60 μg/hour.

27. The system of claim 19, wherein the nitroglycerin-containing composition provides a delivery rate of nitroglycerin at the drug delivery zone at from about 1 μg/cm²/day to about 600 μg/cm²/day.

28. The system of claim 19, wherein the drug delivery zone has a shape selected from the group consisting of a rectangle, a triangle, a circle, and an oval.

29. The system of claim 19, wherein when the dressing is applied to a skin surface to secure a catheter in a vessel the drug delivery zone overlays the vessel upstream from catheter.

30. The system of claim 19, wherein the catheter and the adhesive dressing are co-packaged as part of a kit.

31. The system of claim 19, wherein the catheter and the dressing are integrated into a single device.

32. A method of cathertizing a subject, comprising:
a) placing a catheter in a vessel of the subject; and
b) securing the catheter with an adhesive dressing, wherein the adhesive dressing includes a nitroglycerin-containing composition which delivers a dose of nitroglycerin transdermally to the subject, and wherein the dose of nitroglycerin prevents or reduces the occurrence of phlebitis.

33. The method of claim 32, wherein the adhesive dressing has an area where the adhesive dressing contacts the skin surface, said area being from 50 cm² to 100 cm².

34. The method of claim 32, wherein the nitroglycerin is present in the nitroglycerin-containing composition in an amount of from 6 wt % to 20 wt %.

35. The method of claim 32, wherein the catheter and adhesive dressing are configured to be used for a period of from 4 hours to 7 days.

36. The method of claim 32, wherein when the adhesive dressing is applied to a skin surface of the subject such that the nitroglycerin-containing composition overlays the vessel upstream from catheter.
